(19) United States
(12) Patent Application Publication
(10) Pub. No.: US 2023/0238088 A1
(43) Pub. Date: Jul. 27, 2023

(54) SYSTEM AND METHOD FOR RAPID RESULTS AND REPORTING OF DIAGNOSTIC TEST RESULTS

(71) Applicant: Abbott Rapid Diagnostics Informatics, Inc., Charlottesville, VA (US)

(72) Inventor: Razvan Dima, Davie, FL (US)

(21) Appl. No.: 18/043,509

(22) PCT Filed: Aug. 30, 2021
(86) PCT No.: PCT/US2021/048177

§ 371 Date: Feb. 28, 2023

Related U.S. Application Data
(60) Provisional application No. 63/072,758, filed on Aug. 31, 2020.

(51) Int. Cl. G16H 10/40

(52) U.S. Cl. CPC G16H 10/40

(57) ABSTRACT

System and method for reporting test results for a sample. In an example implementation, patient information is received from a data entry interface. A patient identifier is generated by a software application operating on the data entry interface and associated with the patient information. The patient identifier and a test result generated by a point of care (POC) test instrument used to test a sample provided by the patient corresponding to the patient identifier are communicated to the system. The test result and the patient identifier are correlated with the patient information corresponding to the patient identifier. The data entry interface may be a mobile device having a barcode generating application for generating the patient identifier as a barcode. The data entry interface may also be implemented as a portal operating as an application on a computing device.

### CLAIM OF PRIORITY

This patent application claims the priority of U.S. Provisional Patent Application Ser. No. 63/072,758, titled System and Method for Rapid Results and Reporting of Diagnostic Test Results,” filed on Aug. 31, 2020, the contents of which are incorporated herein by reference in its entirety.

### FIELD OF THE DISCLOSURE

This disclosure relates generally to automated reporting of diagnostic testing data, such as data related to COVID-19 patent testing data, to governmental health agencies.

### BACKGROUND

In recent years, there has been a rise in outbreaks of infectious diseases (e.g., viral diseases, bacterial diseases, etc.) such as the COVID-19 virus, Ebola virus, H1N1pdm09 virus, Middle East respiratory syndrome coronavirus (MERS-CoV), and

Severe Acute Respiratory Syndrome (SARS), to name a few. These infectious diseases are often contagious and easily transmitted from person-to-person in close proximity or through indirect contact via objects and surfaces. To curb the spread of infectious diseases and promote a rapid public health response to infectious disease pandemics, governmental public health agencies have mandated reporting of patient test results that include standardized test results, relevant demographic details, and additional information. An example of such governmental mandate is the United States' Coronavirus Aid, Relief, and Economic Security (CARES) Act.

### SUMMARY

In view of the above, a method of reporting test results is provided. The method in an example implementation includes inputting patient information into a data entry interface. A patient identifier is generated by a patient data entry application. The patient identifier is associated with the patient information. The patient identifier is transmitted from the patient data entry interface to a point of care (POC) test instrument, which is not directly connected to a laboratory information management system. The patient identifier is transmitted along with the patient information from the data entry interface to a cloud-based database which is remote from the laboratory information management system. The patient identifier is received at the POC test instrument from the data entry interface and the sample is tested on the POC test instrument to generate test results. The test results are transmitted along with the patient identifier from the POC test instrument to the cloud-based database. The cloud-based database then marries the test results with the patient information that are associated with the same patient identifier.

In one aspect, the data entry interface is a mobile device or a portal on a computer.

In another aspect, the patient identifier is represented by a barcode generated by the application, and the remote instrument receives the patient identifier by scanning the barcode from a screen of the data entry interface.

In another aspect, the data entry interface is a mobile device comprising a scanning device. The step of inputting the patient information includes scanning an identification card associated with the patient and having at least a subset of the patient information encoded on the identification card.

In another aspect, a method is provided for reporting test results for a sample. Patient information is input into a software application in communication with a remote server. A patient identifier is generated by the application. The patient identifier is associated with the patient information. The patient identifier is transmitted to a POC test instrument. The patient identifier may be transmitted along with the patient information from the software application to a remote server. A test may be run on a biological sample at the diagnostic instrument to generate a test result. The test result and the patient identifier are transmitted from the instrument to the remote server. The remote server correlates the test result and the patient information with the same patient identifier.

In another aspect, a report is generated containing information related to the test results and patient information to be transmitted to a public health agency.

In another aspect, a method is provided for reporting test results for a sample. Patient information is received from a data entry interface. A patient identifier is generated by a software application operating on the data entry interface.

where the patient identifier is associated with the patient information. The patient identifier and a test result generated by a POC test instrument used to test a sample provided by the patient corresponding to the patient identifier are received and the test result and the patient identifier are correlated with the patient information corresponding to the patient identifier.

In another aspect, the method includes generating report information related to the test results and patient information to be transmitted to a public health agency.

In another aspect, a system for reporting test results for a sample includes a remote data collection system comprising a processor, a data storage system, and a communication interface for communicating over a communications interface. At least one data entry interface is configured to display queries for entry of patient data and to generate a patient identifier associated with the patient data, and to communicate the patient data to the remote data collection system. A patient data reporter includes computer program components configured when executed by the processor to:
receive a patient test result from a point-of-care (POC) test instrument and the patient identifier associated with a patient sample corresponding to the patient test result when the patient sample is tested by the POC test instrument;
associate the patient test result with the patient data corresponding to the patient identifier;
collect selected patient data for reporting to a public health agency; and
report the patient test result and corresponding selected patient data to the public health agency by transmitting the patient test result and corresponding selected patient data over the communication network to a communication interface of the public health agency.

In another aspect, at least on data entry interface includes at least one mobile device.

In another aspect, at least one mobile device includes a smartphone, a laptop, or a tablet computer.

In another aspect, the mobile device includes a barcode generator configured to generate the patient identifier in the form of a barcode.

In another aspect, the mobile device includes a scanning device configured to scan a personal identification card having encoded personal data, and to format the encoded personal data as at least a subset of the patient data.

In another aspect, at least one data entry interface includes at least one computing device configured to operate a portal to the remote data collection system.

In another aspect, the portal operating on the at least one computing device includes a user account generator configured to organize data entered by a user into a user account, where each user having a user account performs patient data entry for patients providing samples for testing on the POC test instrument.

In another aspect, the portal includes a user listing component configured to display a list of users having user accounts on the portal. The list includes data relating to patient samples corresponding to the patient data for each patient for which samples are collected and data relating to test results for the samples collected.

In another aspect, the portal includes a patient data component configured to display a patient data screen comprising the patient ID, a test status, and the test results if available when the patient data screen is displayed.

In another aspect, the patient data component is further configured to display the patient's name, birthdate, and test location.

In another aspect, the portal includes a home screen for each user having a user account. The home screen is configured to display selected performance data corresponding to the user account and selected functions with graphical user interface selectors configured to invoke the selected function when selected by the user.

In another aspect, the collection of selected patient data and the reporting of the selected patient data with the corresponding test result are performed automatically when test results are received by the remote data collection system.

### BRIEF DESCRIPTION OF THE DRAWINGS

FIG. 1A is a schematic diagram of an example public health reporting system constructed in accordance with the teachings of this disclosure.

FIG. 1B is a schematic diagram illustrating one example of a workflow that may be followed using the system of FIG. 1A.

FIG. 1C is a schematic diagram illustrating an example of a workflow from a customer administrator view that may be followed using the system of FIG. 1A.

FIG. 2 is a flowchart illustrating operation of an example method for reporting test results and patient data to public health authorities.

FIG. 3A is a flowchart illustrating operation of an example data input system of the public health reporting system in accordance with the teachings of this disclosure.

FIG. 3B-3D illustrate screenshots that may be used as a mobile interface for entering patient data corresponding to a patient providing a sample.

FIG. 3E-3G illustrate screenshots that may be used as a portal interface for entering patient data corresponding to a patient providing a sample.

FIG. 4 is a flowchart illustrating operation of an example diagnostic instrument system of the public health reporting system constructed in accordance with the teachings of this disclosure.

FIG. 5 is a flowchart illustrating operation of an example remote server system of the public health reporting system constructed in accordance with the teachings of this disclosure.

FIG. 6 is a flowchart illustrating operation of an example remote data collection system for use with the public health reporting system constructed in accordance with the teachings of this disclosure.

### DETAILED DESCRIPTION

Disclosed herein are example apparatus, systems, methods, and articles of manufacture that enable entities or persons to comply with public health reporting laws for diagnostic tests. Example implementations disclosed herein may use diagnostic test devices, including automated diagnostic instruments, for providing test results. In particular, the diagnostics test devices may be point-of-care (“POC”) diagnostic testing devices where a diagnostic test is performed at or near the location where the sample was gathered from the user. Such locations may include for example, an in-field testing facility, a medical office or clinic, a patient's home, or ad hoc testing sites such as test sites built to address an emergency such as the COVID-19 pandemic. Ad hoc testing sites may include, for example, testing sites that may be setup in airports, in parking lots, at event venues, or other locations that present risks for which testing is necessary. It is noted that the COVID-19 pandemic and testing for the SARS-CoV-2 virus are used as an example background and pathogen for example implementations. Example implementations of the systems and methods described herein may also be used to address other emergencies involving other pathogens or types of pathogens.

The POC diagnostic instruments used to determine if patients' samples carry the SARS-CoV-2 virus are able to provide results in a short period of time. A remote data collection system is also leveraged to collect patient demographic data, answers to certain patient questions, in addition to the usual identifying information to overcome bottlenecks in the testing process and the mandated public health authority reporting process.

In an example implementation, each patient can be registered, or processed for a test order, at a test site using a remote data collection system. In one embodiment, the remote data collection system includes an interface for a user to input patient information that is stored at a remote server. The remote data collection system is also operable to receive diagnostic test results and store those test results at the remote server. In some embodiments, the remote data collection system is a reporting system that collects patient and diagnostic test data outside of a conventional laboratory information management system (LIMS). In other embodiments, the remote data collection system uses a LIMS system for data collection and reporting. In one example implementation, the remote data collection system may be configured to operate as a cloud service. The remote data collection interface for inputting patient data may be a mobile interface (“mobile interface”) configured to operate on a handheld device, such as for example, a smartphone (e.g. iPhone, etc.), a tablet device, or other similar handheld device. The interface to the remote data collection may also be a portal to the remote data collection system operating using, for example, a browser, which may then operate on any suitable computing device such as a desktop computer or a laptop or a smartphone. A test site may be any location having at least one POC diagnostic instrument configured to communicate with the remote data collection system, and an operator having at least one remote data collection interface to the remote data collection system. The embodiments described herein may be used in traditional diagnostic testing settings, such as for example, physicians' offices and diagnostic laboratories. In response to the high demand for testing created by the COVID-19 pandemic, the present invention may also be used with test sites that may now include pharmacies, department stores, and other ad hoc test sites of the type described above.

As noted, the patient data is collected using the remote data collection interface, which may be a mobile interface or a portal. The mobile interface (or portal) may be configured to prompt the operator for certain patient information (described in more detail below), which may include demographic information and any other type of information mandated by public health authorities. The mobile interface (or portal) may also be configured to generate a unique patient identifier that is associated with a particular patient's inputted data. The unique patient identifier distinguishes a patient data input entry from other patient data input entries and may take the form of a code or set of digits that is unique from any other code or set of digits in the remote data collection system. In one example, the patient identifier may be generated as a barcode, a 2-D barcode or a QR code. Other examples may use other types of codes or combinations of codes including, for example, other machine-readable codes.

In one embodiment, at least some data entry may be performed in an automated manner. In one example of this, certain jurisdictions issue identification cards, such as driver's licenses, that contain a machine-readable code, such as a barcode, that incorporates the card holder's identification information, such as name and address. The mobile interface may also interface with a camera or other optical reader to image the machine-readable code to scan demographic and patient information from an identification card having such information encoded to advantageously speeding the process of collecting the mandated information. In some examples, such as where a test site is connected to a LIMS, some or all of the patient data or demographic information may be available to the remote data collection where certain data was collected by healthcare providers at the physicians' offices for example. The mobile interface or portal may be configured to retrieve the patient data and demographic information electronically, associate it with the patient identifier, and communicate the patient data and demographic information with the patient identifier to the remote data collection system. Although the above data input process has been described in relation to a mobile interface, the above process can also be used with a portal.

During the patient registration process at the test site, the operator also takes a patient sample. The POC diagnostic instrument may be configured to input the patient id as an identifier corresponding to the patient's test results when the patient sample is loaded in the POC diagnostic instrument to run the test. In one implementation, the POC diagnostic instrument may include a barcode reader, which may be used to scan the patient identifier barcode from the mobile interface on the handheld device.

The mobile interface may be configured to upload the patient's data (including demographic data, and responses to questions to which public health authorities desire answers) to the remote data collection system. In one example, the patient data and patient identifier are uploaded independently of the communication of the patient identifier and the test results by the POC diagnostic instrument. The remote data collection system would then correlate the independently received patient data and test results. In another example, the mobile interface or the portal may include a data verification procedure in which the remote data collection interface checks the patient data to ensure that all mandated data for reporting is captured before a patient identifier is generated. The patient identifier may then be provided to the POC diagnostic instrument to identify the patient's test results once the patient's sample has been tested.

The remote data collection system may be configured to receive the patient data identified by the patient identifier from the mobile interface, and the patient test results identified by the patient identifier from the POC diagnostic instrument. The remote data collection system may correlate the patient data with the patient's test results based on the patient identifier. The patient data and test results may then be formatted for communication to the public health authorities. The remote data collection system may be implemented as mentioned above as a cloud service, which may then easily and securely transfer information to the public health authority database systems. As used herein, communication to the public health authority may be directly to the public health authority or may be through one or more intermediaries that delivers the results to the public health authority. In one example, the reporting to a public health agency includes the process of sending report data to AIMS (APHL Informatics Messaging Services) which then reports the data to the relevant public health agency. Although the present invention is described several times as communicating information with a public health agency, one of skill in the art will understand that testing and results related information can also be sent to non-public health entities such as other governmental institutions, employers, schools, public and private companies and non-profit organizations. For example, an employer may wish to screen and gather data from employees enrolled in a screening program.

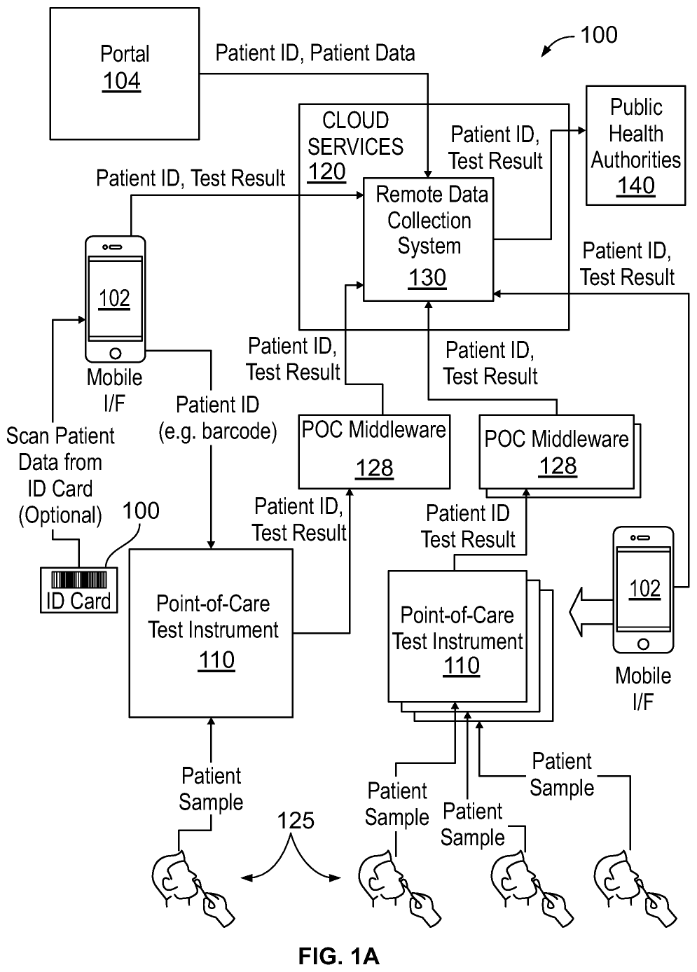

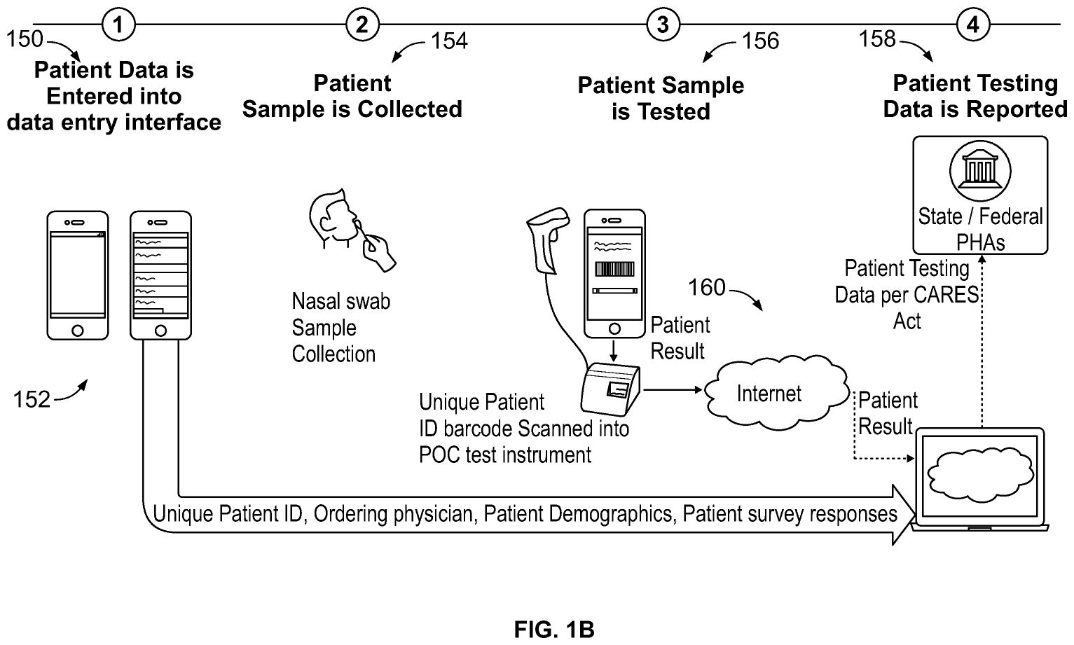

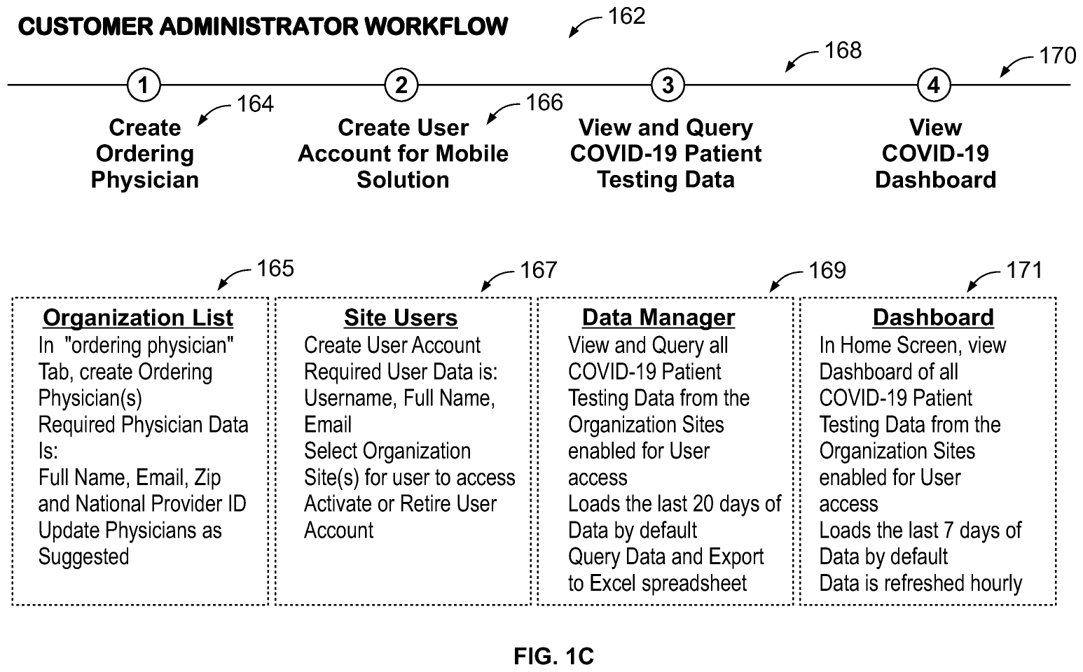

FIG. 1A is a schematic diagram of an example public health reporting system 100 constructed in accordance with the teachings of this disclosure. The system 100 in FIG. 1A includes a test site having a data entry interface, and at least one POC test instrument 110. The data entry interface may be implemented using a mobile device or mobile interface 102 and/or a portal 104. The mobile devices 102, the portal 104, and POC test instrument 110 are each configured to communicate with a cloud-based services system 120 that includes a remote data collection system 130 having an interface to a public health authorities (PHA) information system (directly or through an intermediary as described above) 140.

It is noted that the mobile devices 102, the portal 104, the remote data collection system 130, the cloud services 120, the POC test instruments 110 and the POC middleware 128 operate using at least one processor. Functions described below are understood to be performed by processors executing programmed computer instructions. It is further understood that the public health authorities 140 receive and transmit data over the Internet via servers and other processor-based components. The mobile devices 102 may be processor-controlled devices. The portal 104 may be implemented using a browser page or other programming element configured to communicate over the Internet, where the browser page or other programming element operate on a computer (e.g. laptop, desktop, etc.).

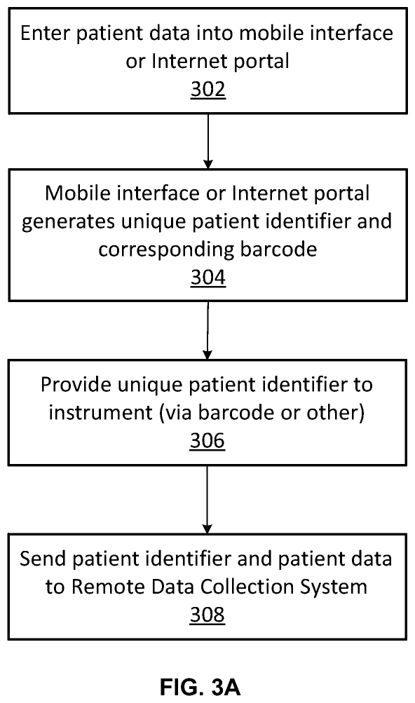

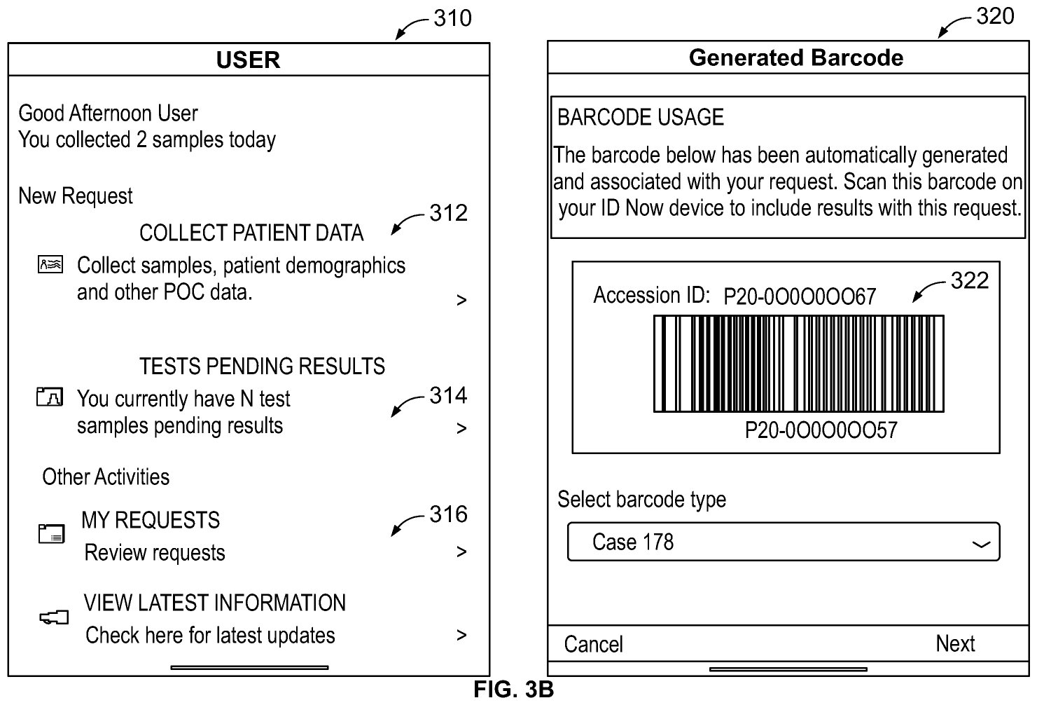

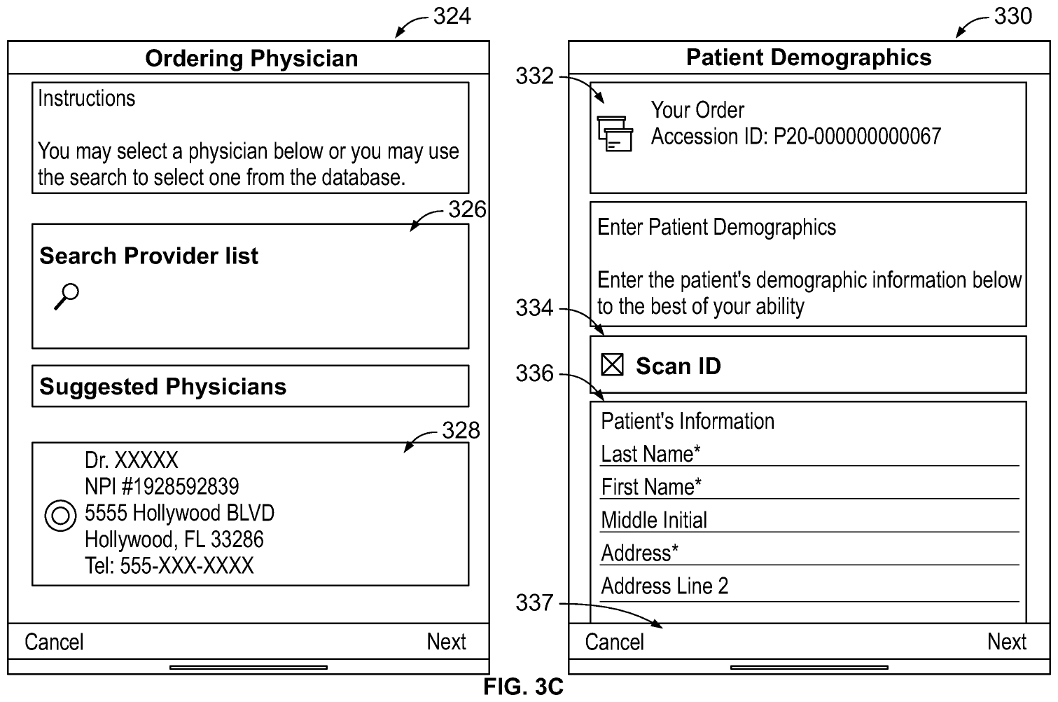

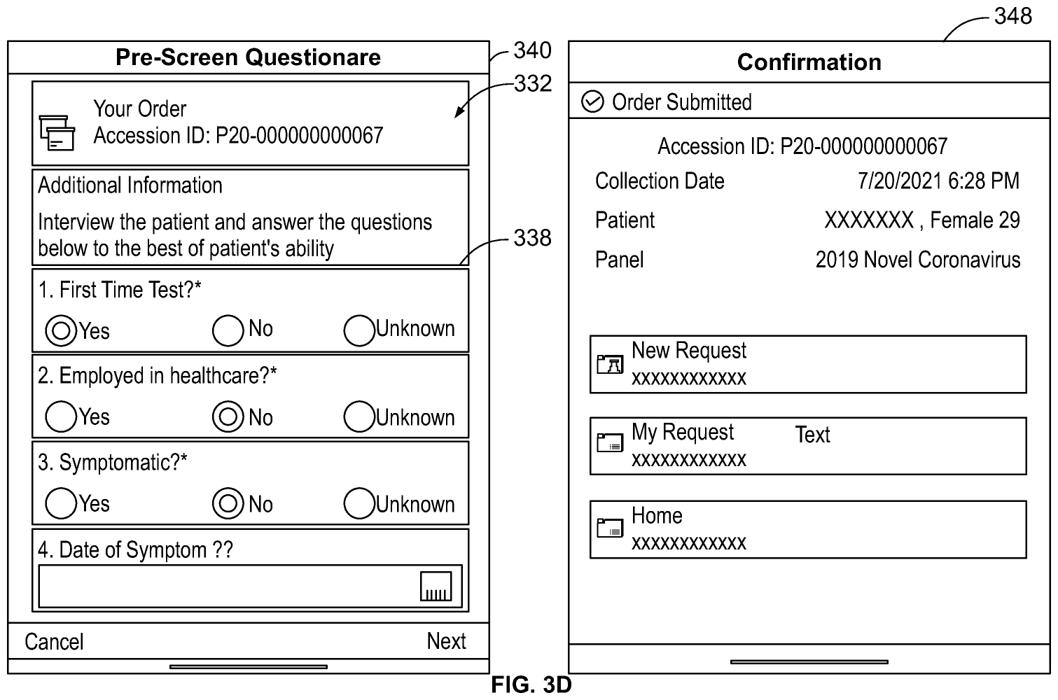

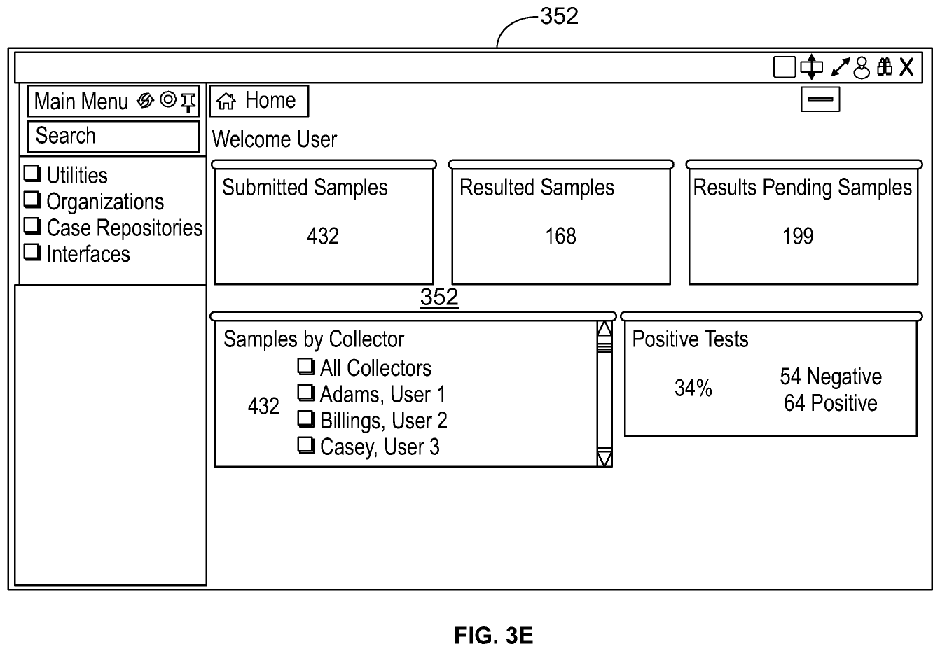

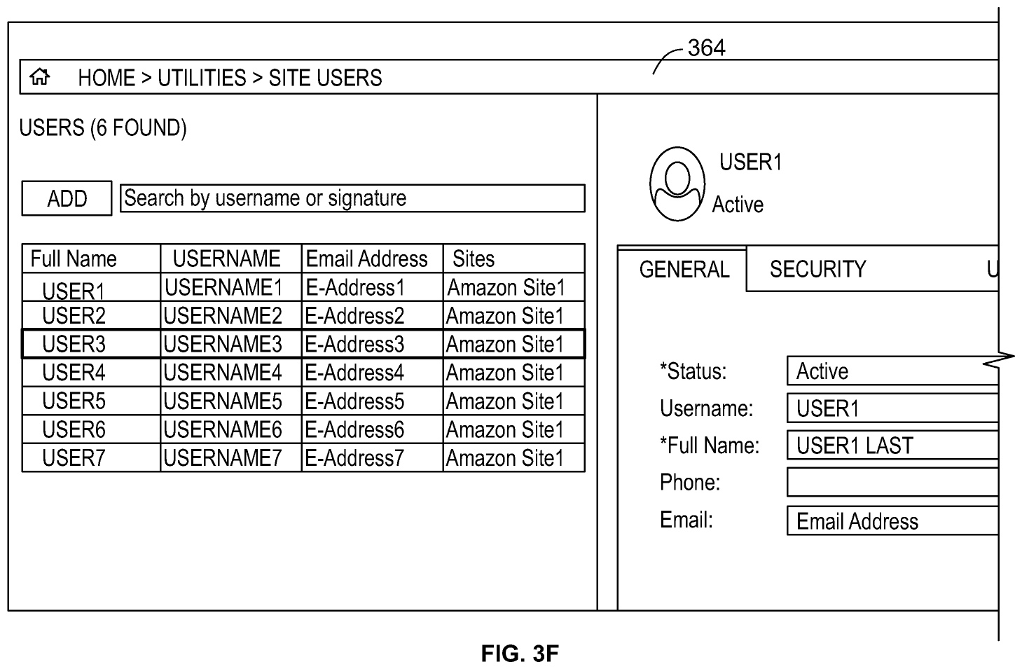

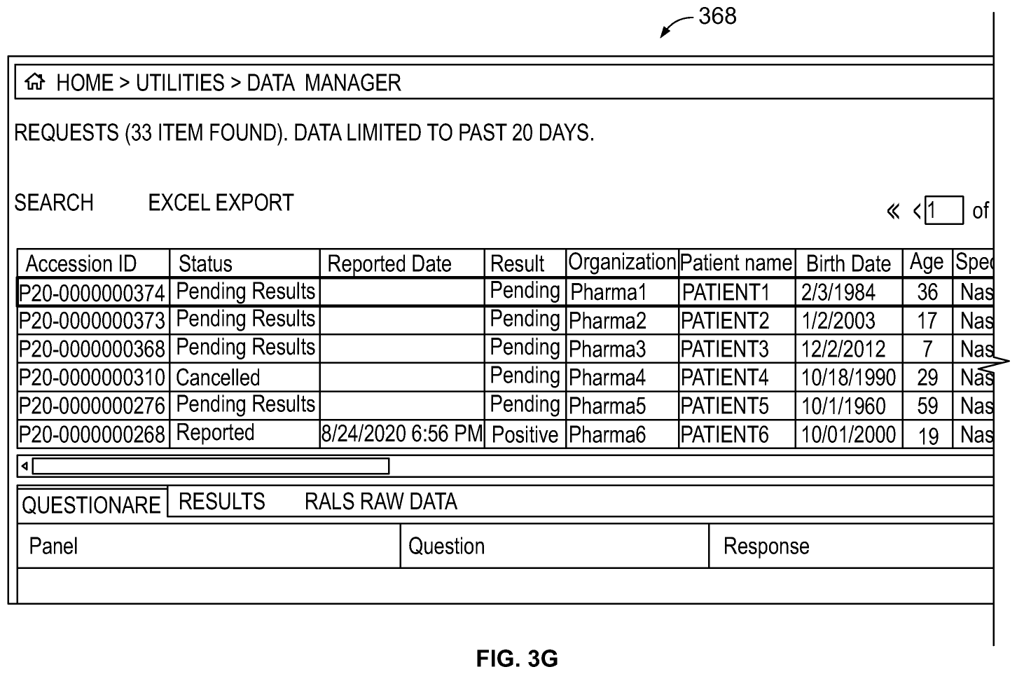

The mobile interface 102 may be implemented as a smartphone or handheld device application having data input screens (examples of which are described below with reference to FIGS. 3B-3D), a patient identifier generator, a data formatting program, and a communications interface for connecting to the remote data collection system 130. The data input screens may be configured as data input forms prompting the operator for information such as name, address, age, etc. The data input screens in an example implementation prompts the operator for data elements to be collected and reported for SARS-CoV-2 laboratory tests, for the transmission of complete laboratory testing data to CDC or the Secretary's designee. Examples of such data for mandated reporting is listed below in Table A.

TABLE A
Examples of Information Requested for Mandated Reporting

Test Ordered
Device Identifier
Test Result
Test Result Date
Accession #/Specimen ID
Patient Age
Patient Race
Patient Ethnicity
Patient Sex
Patient Residence Zip Code
Patient Residence County
Ordering Provider Name and NPI (as applicable)
Ordering Provider Zip
Performing Facility Name and/or CLIA number,
if known
Performing Facility Zip Code
Specimen Source (e.g. provided using
appropriate LOINC, SNOMED-CT, or SPM4
codes or equivalent detailed alternative codes
Date test ordered
Date specimen collected

In some implementations, the data input screens may include prompts for additional information such as for example demographics data elements that should also be collected and reported to state or local public health departments. Such demographic data elements may include information of the type listed in Table B below.

TABLE B
Examples of Demographic Data Elements

Patient name (Last name, First name, Middle
Initial
Patient street address
Patient phone number with area code
Patient DOB
Ordering provider address
Ordering provider phone number

In an example implementation for use in addressing the COVID-19 pandemic, the mobile interface may be configured with data input screens requesting special information. The data fields listed in Table C below are specific to SARS-CoV-2 and considered “ask on order entry” (AOE) questions for traditional Electronic Health Records or Laboratory Information Management Systems. These elements should be collected and be conformant with standard interfaces used to communicate with public health authorities (e.g. HL7 Version 2.5.1 Lab order Interface Implementation Guide):

TABLE C
Examples of COVID-19 specific Data Elements

First test (Y/N/U)
Employed in healthcare? Y/N/U
Symptomatic? Y/N/U and then Date of Symptom Onset mm/dd/yy
Hospitalized? Y/N/U
ICU? Y/N/U
Resident in a long-term care facility (including skilled nursing
facilities, nursing homes, long-term care for the developmentally
disabled, and assisted living homes): (Y/N/U)
Pregnant? Y/N/U P

In implementations at an ad hoc test site, such as for example, in a parking lot under a tent, the operator of the mobile interface 102 may enter the data manually on to the handheld device operating the mobile interface 102. The mobile interface 102 may be deployed for use in a test site in a traditional testing setting, such as for example, a physician's office. In another embodiment, the mobile interface 102 may be configured to interface with a LIMS system to retrieve patient related data. The mobile interface 102 may then retrieve patient data and other data of the type listed in Tables A, B, and C above via such an interface.

The mobile interface 102 may also interface with, or include functions for, a barcode reader to read data from a barcode. In one example, a barcode reader may be used to scan information encoded as barcode on a patient's identification card 150. Many jurisdictions have mandated that identification cards, such as driver's licenses, encode the identifying and demographic information on the identification cards in barcode (e.g. PDF 417 barcodes). The ID card barcode 150 may be used to retrieve a portion of the required data reducing the amount of data that is manually input by the operator.

The patient identifier generator may be a barcode generator, or any other suitable identification generator configured to encode a data element that is used for identification. The patient identifier may be generated in machine readable and barcode formats, which may include, for example, QR codes, Code 128, and 3of9, as some examples.

As noted above, the test site may be in a traditional testing location, which may provide the operator with data connectivity for the mobile interface 102 and the POC test instruments 110. In ad hoc sites, the mobile interface 102 and the POC test instrument 110 may be provided with a Wi-Fi interface to a Wi-Fi access point, cellular access point or other suitable data interface with the Internet in order to connect to the cloud-based services 120. For POC instruments connected in this manner, cloud-based services can include POC instrument related middleware 128 that communicates further with the remote data collection system 130. The POC middleware 128 may be any suitable networking devices that may be used to facilitate communication between the POC instruments 110 and the remote data collection system 120. Multiple POC test instruments 110 may be connected over a network interface (wireless or wired) with a bridge or gateway to the Internet. In one example, RALS™ Link, available from Abbott Laboratories, provides a communication gateway that includes a cellular connection to the Internet, to connect POC instruments to remote servers such as cloud-based servers. Further, in one example, different institutions or companies can use their own POC test instruments 110 to run tests and submit the relevant patient and test data to a public health authority. In this regard, with respect to FIG. 1A, the multiple POC test instruments 110 shown can represent instruments and middleware of different institutions or companies who report their respective data to the remote data collection system 130. In another example, the multiple POC test instruments 110 shown are different locations of the same institution or company who reports its data to the remote data collection system 130.

The POC middleware 128 may include connections to one or more POC instruments 110 at a single testing location. Each POC instrument 110 may be a testing site and one or more POC middleware 128 devices may be provided at a single test location. The POC middleware 128 may be deployed in any suitable location worldwide providing the remote data collection system 130 with the ability to collect patient data and test results from anywhere in the world.

In locations supporting multiple test sites (e.g. multiple POC test instruments, multiple operators, etc.), a portal 104 may be provided as another data entry mechanism, or as an interface for requesting and viewing information regarding the testing location. The portal 104 may be configured to communicate with the POC test instruments 110 used at the testing location and with the remote data collection system 130. The portal 104 may be configured to collect patient data, such as for example, any data in Tables A, B, or C. The portal 104 may also request test results, or quality control information related to the operators or the POC test instruments 110.

The system 100 described above with reference to FIG. 1A allows for the collection of patient data and creation of a patient ID for patient testing in an example remote data collection system. The system 100 provides flexibility in allowing for use in a small clinic environment, an environment that is not a clinic or equipped for medical procedures, or a large patient sample collection and/or testing environment. The flexibility of the system 100 in FIG. 1A may be more fully appreciated by a description of types of workflows available through its use.

FIG. 1B is a schematic diagram illustrating one example of a workflow 150 that may be followed using the system of FIG. 1A. FIG. 1C is a schematic diagram illustrating an example of a workflow from a customer administrator view that may be followed using the system of FIG. 1A. The workflows in FIG. 1B and 1C are described below with reference to components described above with reference to FIG. 1A. Patient data may be collected at 152 using either a mobile device 102 or the portal 104 as described above. On a mobile device 102 data entry may be prompted using data entry user interface screens such as those described below with reference to FIGS. 3B-3D. On a portal 104, similar data entry forms may be presented to the user as described below with reference to FIGS. 3E-3G.

The data entry step at 152 may include prompts or forms for obtaining information such as an order physician name, patient demographics information, and the type of information used by public health agencies, such as for example the type of data collected under the CARES Act mandate. The mobile device 102 may be programmed to generate a patient ID, which may be displayed on the mobile device display in the form of a barcode. Either before, after or contemporaneously with the data entry, a patient sample may be collected at step 154. At step 156, the patient sample may be loaded into a testing system, such as for example, a POC test instrument 110 (in FIG. 1A). The POC test instrument 110 may be equipped with a data entry mechanism for inputting the patient ID, and with a mechanism for associating the patient ID with the patient sample. In one example, the patient ID may be displayed in the form of a barcode and scanned by the POC test instrument 110 to correlate the patient ID with the patient sample. After the POC test instrument 110 generates a result from the test sample, the results are associated with the patient ID for communication to the remote data collection system 130 (in FIG. 1A). The results and patient ID may be communicated over the Internet 160 for reporting to government agencies at 158.

FIG. 1C illustrates a customer administrator workflow 162, which may be used for example in managing an organization-driven or multi-user testing program. At step 164 of the workflow 162 in FIG. 1C, a list of ordering physicians may be created using contact and other information (e.g. data in 165) for physicians that may treat the patients to be tested. At step 166, a user account may be created for each user that is tasked with obtaining information from patients during test procedures. The user may or may not be the person responsible for actually obtaining the patient sample. The user account setup in step 166 may be performed using a user account generator that operates similar to that of any typical user account creating tool for resources available on the Internet. Data block 167 lists the type of data that may be collected for each user. Once an account is created, each user performs patient data collection functions under the user's user account. The data collection under a user account allows for the collection of data for each user. This provides data that may be used for monitoring performance, monitoring consistency in the way tasks are performed, and record-keeping that correlates the patient identifiers of the patients for whom samples are collected with the user that collected the data.

At step 168, the remote data collection system 130 (in FIG. 1A) may provide data viewing functions to allow an administrator or test program manager to query and view patient testing data as described in 169. It is noted that the features indicated in 169 are examples of the type of data viewing possible. Data from more or fewer than 20 days, or a programmable number of days, may be made available. Data may be queried in many ways and exported to any suitable format (e.g. Excel and others).

At step 170, a data dashboard may be provided to provide access to patient data from different organization testing sites. The text block 171 in FIG. 1C indicates parameters for viewing the data. Data may be loaded from (for example) the last 7 days or refreshed hourly (for example). The number of days of data available may be greater than or equal to seven, or selectable. The data may be refreshed at different intervals.

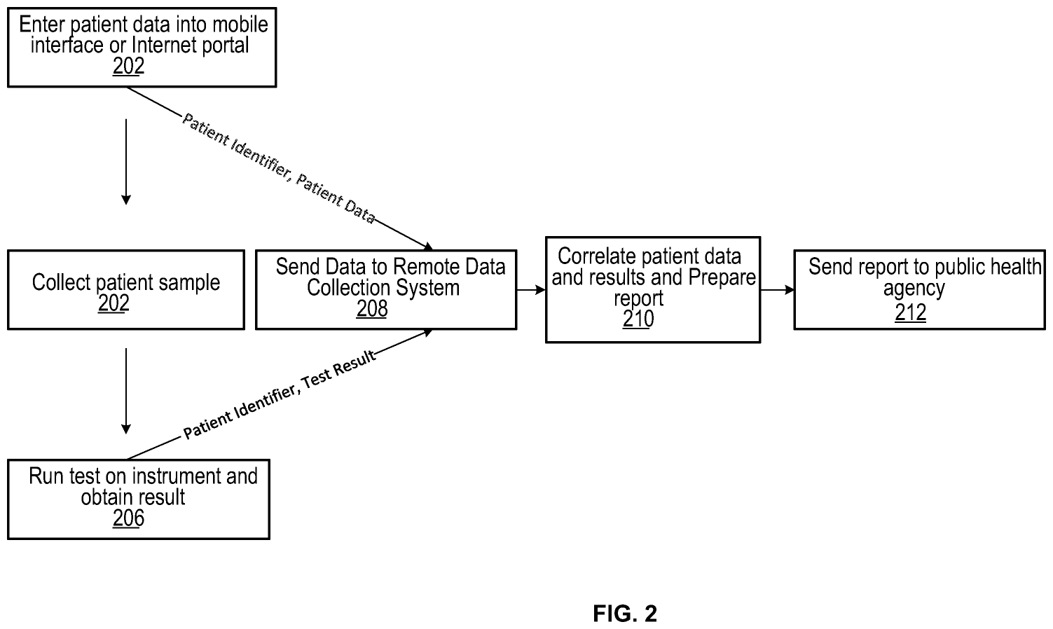

FIG. 2 is a flowchart illustrating operation of an example method 200 for reporting test results and patient data to public health authorities. The method 200 is described with reference to the use of the system 100 in FIG. 1A as a hardware platform for purposes of clarity in the description. FIG. 2 illustrates operation of the system 100 when a patient approaches a testing site for a diagnostic test.

The process may typically begin at step 202 in FIG. 2 when a patient approaches the operator and the operator enters the patient data into the mobile interface 102 or the portal 104. The patient data entry process is described below in more detail with reference to FIG. 3A. In some implementations, the mobile interface 102 may be configured to allow the patient to enter the data him- or herself. Once the patient data, which may be any of the data listed above in Tables A, B, or C, is entered, the patient data is sent to the remote data collection system 130 via a communication interface to the cloud at step 208. At step 204, the operator collects the patient sample, which may depend on the requirements of the POC test instrument 110. With respect to testing for the SARS-CoV-2 virus, the POC test instrument 110 may be of the type that receives patient samples from having swabbed the patient's airways. The POC test instrument 110 may receive patient samples in the manner in which it was configured to operate. At step 206, the operator then operates the test instrument. When the test is completed and results are available, the test results and patient ID may be communicated to the remote data collection system 130 at step 208.

At step 210, the remote data collection system 130 correlates the patient's test results with the patient data collected in step 202. The data and test results are formatted appropriately for reporting to the public health authorities at step 212.

FIG. 3A is a flowchart illustrating operation of an example data input system of the public health reporting system 100 in accordance with the teachings of this disclosure. As shown at step 302 in FIG. 3A, the patient data may be collected using the mobile interface 102 (in FIG. 1) or the portal 104 (in FIG. 1). At step 304, the mobile interface 102 or the portal 104 (FIG. 1) generates a patient ID for an order being entered for the patient's test. The mobile interface 102 or portal 104 may include a program or an interface to a program that generates the patient ID as a barcode, or the patient ID may be encoded in another suitable format.

The mobile device 102 (in FIG. 1) that may be used for the mobile solution for collecting patient data may be programmed to present a variety of different user interfaces to solicit specific information from the patient, or for enabling the user to perform certain functions. The mobile device 102 may provide a home screen 310, such as the screen 310 shown in FIG. 3B. The home screen 310 may provide the user with a collect patient data button or selector 312 to initiate a patient data collection process when selected by a user. The patient data collection process may involve displaying screens for entering data such as the screens described with reference to FIGS. 3B-3D. A pending results button 314 may be selected to be selected to access records for patients whose data has been entered but are still awaiting test results. The pending results button 314 may provide data entry or modification functions for when patient data entry may not be complete or may require revision before the results are ready. The home screen 310 may provide functions in an area of the screen 316 for viewing data or for modifying how the data is organized.

When patient data is to be entered, a patient ID may be generated during the data entry process. The patient ID may be displayed on a barcode generation screen 320 as a barcode 322.

In FIG. 3C, part of the patient data entry process may include entering an ordering physician identifier using, for example an ordering physicians' screen 324. The screen 324 may include a search tool 326, and a results section 328. A patient demographics form 330 may be presented to enter the patient data as corresponding to a patient ID 332. The mobile device 102 (in FIG. 1A), may be programmed to invoke a scanning device (e.g. a camera) to scan an identifier, such as an ID card with patient data programmed in a scan code or bar code. The scanning function may be invoked by a user by a press of a scan button 334.

The user may also be presented with a form 336 for entering the requested data. Data may be entered directly into the phone. Alternatively, the scanning device may be programmed to receive the scanned data and fill the patient form with the scanned data. The screen 330 may include basic user interface navigation controls, such as a next and previous button to proceed to another section of the form. For example, the user may press a Next button 337 to proceed to another form, such as a pre-screen questionnaire form 340 in FIG. 3D. Some information may be entered into the form as not all information is necessarily available on an identification card. An additional information form 338 may be used to enter additional information, such as for example, employment information, demographic information, etc. A confirmation screen 348 may be presented at the end of the data entry process. The confirmation screen 348 may include the patient ID, name, test panel, age, etc.

FIGS. 3E to 3G depict screens that may be displayed when using a portal for data entry. FIG. 3E illustrates a home screen 350 for a user. The user may be prompted to log in first using a typical log in procedure with username and password. The home screen 350 may display certain data for the user 352 and provide functions or buttons for the user to select to view different data.

The portal functions may include administrative functions. For example, the portal may include a data manager mode that uses a user listing component to allow the user to view the data entered by other users. The site user data screen 364 in FIG. 3F illustrates an example of a screen that lists the users having an account on the remote data collection system. The users may be displayed with usernames, email addresses, and site information, for example. Details for each user may be displayed when selected.

The data manager may also display a patient data screen 368 generated by a patient data component for listing each patient that has had patient data entered as shown in FIG. 3G. The screen 368 may display the patient ID, the status (e.g. pending results, canceled, reported, etc.), a reporting date if reported, the test results, an organization if relevant, the patient's name, birthdate, and any other item of information that may be relevant.

Referring back to FIG. 3A, at step 306, the operator may provide the patient ID in encoded format to the POC test instrument 110 (FIG. 1). The POC test instrument 110 includes a barcode reader, or an alternative code reading device capable of decoding the encoded patient ID. The POC test instrument 110 correlates the patient ID with the sample loaded into the POC test instrument 110 for testing. At step 308, the patient identifier and the patient data consistent with reporting requirements is communicated to the remote data collection system. It is noted that the patient data consistent with reporting requirements may be communicated with the patient data at any time during the data entry process. For example, the mobile interface 102 may operate as a form such that the data is received at the mobile interface and communicated to the remote data collection system as it is entered. Alternatively, the patient data may be formatted and communicated to the remote data collection system when the patient data has been collected. Regardless of when or how the patient data is communicated, the patient identifier identifies the patient data to correspond with test results identified by the same patient identifier provided to the POC instrument at step 306.

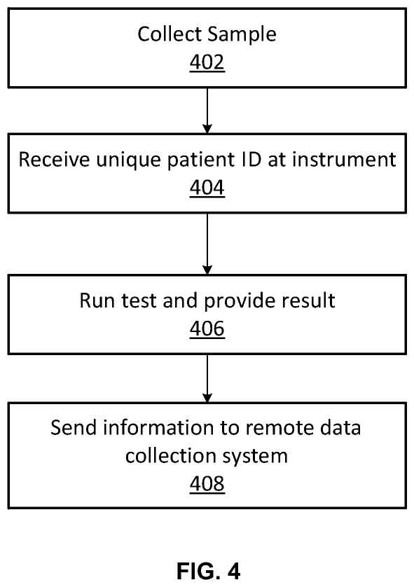

FIG. 4 is a flowchart illustrating operation of an example diagnostic instrument system of the public health reporting system 100 constructed in accordance with the teachings of this disclosure. The POC test instrument 110 (in FIG. 1) may be any suitable diagnostic test system configured to test patient samples for the pathogen of interest, such as for example the SARS-CoV-2 virus. The operator collects the sample at step 402. The sample collection may be performed by swabbing the area of interest on the patient's body, collecting blood or other bodily fluid, or by taking a measurement of a body metric. The sample collection step depends on the capabilities of the POC test instrument being used.

At step 404, the POC instrument receives the patient ID entered into the POC test instrument at step 306. In one example, the patient ID is read by a barcode reader connected to the POC test instrument. In other examples, the patient ID may be received via manual entry using, for example, a keypad or touch display panel, if one is part of the POC test instrument. The POC test instrument is configured to correlate the patient ID with the test sample for which an assay is to be run. At step 406, the POC test instrument is controlled to run the assay, and to provide a test result. The test result may be displayed or provided in a message for display on the mobile device used for the mobile interface, or on the portal. The test result is also correlated with the patient ID and communicated to the remote data collection system 130 (in FIG. 1) via a POC middleware using the communication systems described herein.

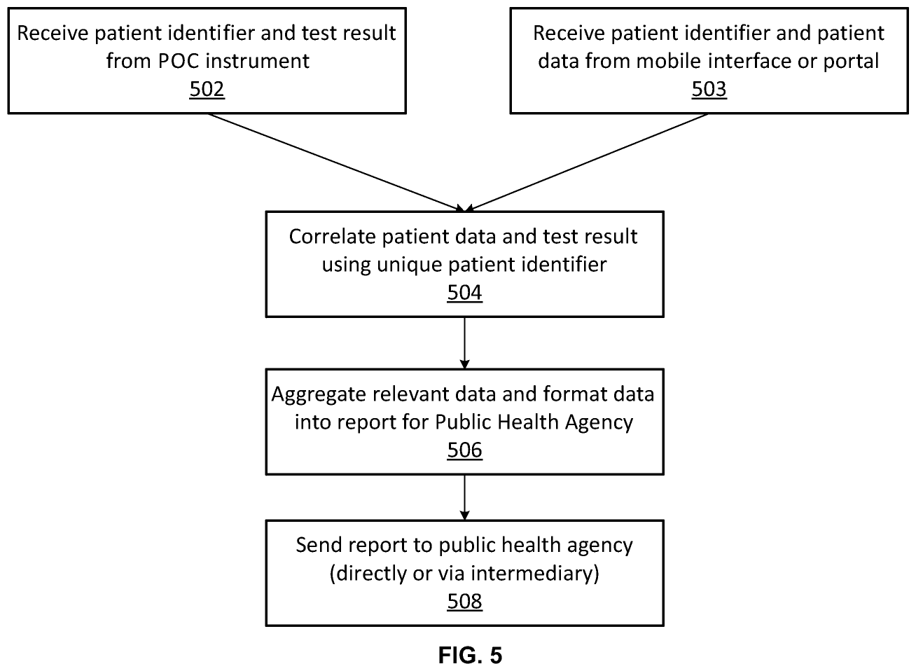

FIG. 5 is a flowchart illustrating operation of an example remote server system of the public health reporting system 100 constructed in accordance with the teachings of this disclosure. As noted above, the remote data collection system 130 may be configured to operate on one or more computing devices having a network interface, memory and a database storage system, and one or more processors. The computing devices may be servers or networked computers, or devices operating as a cloud-based service. In one embodiment, the data collection system is cloud-based laboratory. In one embodiment, the data collection system is separate from the data storage of a LIMS system. In other embodiments, the data collection system leverages an existing LIMS system. In each case, the remote data collection system 130 is configured to receive at step 502 test results from the POC test instruments and patient data from the mobile interface 102 or the portal 104 (in FIG. 1). The remote data collection system 130 may include a data receiver configured to receive the test results from the POC test instruments identified by the patient ID and the patient data from the mobile interface 102 or portal 104 identified by the patient ID. The remote data collection system may receive the patient identifier and the patient data at step 503 in FIG. 5.

It is noted that the example described here with reference to FIG. 5 refers to patient data as the patient data communicated by the remote data collection system to meet any reporting requirements as described above with reference to Tables A, B, and C. In addition, while example implementations are described with emphasis on reporting requirements in addressing the COVID-19 pandemic, specific reporting requirements may be instituted for other emergencies that may require different data to be collected and reported with test results for other relevant tests.

The data receiver may correlate the patient data with the test results based on the patient identifier at step 504. At step 506, the remote data collection system may identify and format the specific patient data elements to be reported and aggregate the selected patient data with the test result corresponding to the patient identifier. The data may be formatted into a report in any form suitable for use by the public health agency that requires the data. At step 508, the report may be communicated to the public health agency electronically. As described above, communication to the public health authority may be directly to the public health authority or may be through one or more intermediaries that delivers the results to the public health authority. In one example, the reporting to a public health agency includes the process of sending report data to AIMS (APHL Informatics Messaging Services) which then reports the data to the relevant public health agency.

The present invention also includes a data manager portal that allows managers associated with the remote data collection system 130 to manage the system and view reports associated with the operation and results of the system. In this data manager portal, a manager of the system creates and manages users of the system, create and manage approved instruments for the system and populate some data available to a user such as ordering physician information. The manager may also be able to view and query patent testing data. For COVID-19 data for example, the manager may view & query all COVID-19 patient testing data from the organization sites enabled for manager and/or user access, loads the last 20 days of data by default, query data and export to Excel spreadsheets. The manager may also be able to view a dashboard that provides summaries of patient and/or testing data. For COVID-19 data for example, the manager may view a dashboard of all COVID-19 patient testing data from the organization sites enabled for manager and/or user access, load the last seven days of patient and/or test data by default and view such data refreshed hourly.

In some examples, the present invention provides a reporting format for PHA for traditional LIMS systems. In this case, the LIMS system aggregates the relevant patient and test data and puts it into an appropriate report format for public health reporting. The PH data can then be conveniently provided to the PHA by sending PH report data from the LIMS system to the remote data collection system which then transfers the information to the PHA.

In example implementations, a convenient conduit and system for reporting data to PHA's are provided as described herein. In this regard, patient and test data information can be received by the remote data collection center from sources other than as described above, but example implementations of systems and methods described herein can aggregate, format and/or send the data to a PHA as described herein. In some examples, a reporting format is provided for PHA for traditional LIMS systems. In this case, the LIMS system aggregates the relevant patient and test data and puts it into an appropriate report format for public health reporting. The PH data can then be conveniently provided to the PHA by sending PH report data from the LIMS system to the remote data collection system which then transfers the information to the PHA. In other examples, example systems and methods described herein provide a reporting format for PHA for traditional LIMS systems, such as those in hospitals and hospital healthcare systems, in an additional manner. In this case, the remote data collection center receives patient and test data information from the hospital or hospital healthcare system, aggregates the relevant patient and test data, puts it into an appropriate report format for public health reporting and then transfers the information to the PHA. In this case, the patient and test data may already be correlated with each other by the LIMS prior to sending the data to the remote data collection center or the remote data collection center may correlate the data. In another example, patient and test data information can be received from other POC testing than that described herein or from in-home testing. In both cases, patient and test data information can be collected from POC or in-home testing systems and stored in a database. The remote data collection center receives patient and test data information from the database that may already have been correlated with each other, aggregates the relevant patient and test data, puts it into an appropriate report format for public health reporting and then transfers the information to the PHA. In this case, the patient and test data may already be correlated with each other by the database prior to sending the data to the remote data collection center or the remote data collection center may correlate the data.

The disclosure provided herein describes features in terms of preferred and exemplary embodiments thereof. Numerous other embodiments, modifications and variations within the scope and spirit of the appended claims will occur to persons of ordinary skill in the art from a review of this disclosure.

1.-20. (canceled)

21. A system comprising:
a remote server;
a data entry application including a data entry interface to be presented on a display screen of a mobile device;
computer-readable instructions; and
one or more processors to execute the computer-readable instructions to:
cause, via the data entry application and responsive to receipt of patient information for a patient via the data entry interface, a patient identifier to be generated;
cause the mobile device to transmit the patient identifier and the patient information to the remote server;
cause a point-of-care test device to transmit the patient identifier and a test result generated for the patient by the point-of-care test device to the remote server from the point-of-care test device; and
associate, at the remote server, the patient information and the test result based on the patient identifier.

22. The system of claim 21, wherein the one or more processors are to cause the remote server to transmit the test result and at least a portion of the patient information to a public health agency.

23. The system of claim 21, wherein the one or more processors are to cause the test result to be displayed at the mobile device via the data entry interface.

24. The system of claim 21, wherein the patient identifier includes encoded data.

25. The system of claim 24, wherein the patient identifier includes a barcode or a QR code.

26. The system of claim 21, wherein the remote server is separate from a laboratory information management system.

27. A system comprising:
a patient data intake application executed on a mobile device, the patient data intake application to:
receive patient information for a subject;
generate a patient identifier responsive to the receipt of the patient information, the patient identifier different than the patient information; and
output the patient identifier and the patient information for transmission via one or more communication networks;
a test device to:
access the patient identifier;
perform a test involving a biological sample from the subject;
generate a test result for the test;
output the patient identifier and the patient information for transmission via the one or more communication networks; and
a remote server in communication with the mobile device and the test device via the one or more communication networks, the remote server to:
correlate the patient information with the test result based on (a) the patient identifier and the patient information received from the patient data intake application and (b) the patient identifier and the test result received from the test device; and
transmit the test result and at least a portion of the patient information for receipt by a public health agency via the one or more communication networks.

28. The system of claim 27, wherein the test result is a first test result and the remote server is to:
aggregate the first test result, the patient information, and a second test result for the subject to generate aggregated data for the subject; and
transmit the aggregated data to the public health agency.

29. The system of claim 27, wherein the remote server is to adjust a format of one or more of the patient information or the test result for transmission to the public health agency based on a reporting format associated with the public health agency.

30. The system of claim 27, wherein the test device is to access the patient identifier via a scanner of the test device, the scanner to scan an image of the patient identifier presented by the patient data intake application.

31. The system of claim 27, wherein the patient data intake application is to output the test result for display at the mobile device.

32. The system of claim 27, wherein the test result is a first test result and the test device is to associate the first test result and a second test result with the patient identifier.

33. The system of claim 27, wherein the subject is a first subject, the test result is a first test result, the patient identifier is a first patient identifier, and the remote server is to:
correlate patient information for a second subject with a second test result for the second subject based on a second patient identifier generated by the patient data intake application for the second subject; and
transmit a report for receipt by the public health agency, the report including (a) the first test result and at least a portion of the patient information for the first subject and (b) the second test result and at least a portion of the patient information for the second subject.

34. A method comprising:
generating, at a mobile device, a patient identifier for a patient, the patient associated with patient information received via a data entry application on the mobile device;
transmitting, via the mobile device, the patient identifier and the patient information to a cloud-based database;
detecting, at a test device for a biological sample, the patient identifier;
transmitting, via the test device, the patient identifier and a test result for the patient to the cloud-based database; and
correlating, at the cloud-based database, the test result with the patient information based on the patient identifier.

35. The method of claim 34, further including causing, via the test device, the test result to be displayed by the data entry application.

36. The method of claim 34, wherein the detecting of the patient identifier at the test device is based on a scanned image of the patient identifier.

37. The method of claim 34, further including:
modifying, at the cloud-based database, a format of the patient information to generate formatted patient information; and
generating aggregated data including the test result and at least a portion of the formatted patient information.

38. The method of claim 37, further including transmitting, via a communication network, the aggregated data for receipt by a public health agency.

39. The method of claim 37, wherein the test result is a first result and the aggregated data includes a second test result for the patient.

40. The method of claim 34, wherein the patient identifier is a first identifier associated with the patient and further including detecting, from a second identifier associated with the patient and at the mobile device, at least a portion of the patient information based on data encoded in the second identifier.
